(19) United States
(12) Patent Application Publication
(10) Pub. No.: US 2015/0148789 A1
(43) Pub. Date: May 28, 2015

(54) METHOD OF POSITIONING A SURGICAL LASER FIBER AND SURGICAL LASER FIBER POSITIONING ARRANGEMENT INCLUDING A FLEXIBLE INTRODUCER SLEEVE FOR IMPLEMENTING THE POSITIONING METHOD

(71) Applicants: Joe Denton Brown, Panama City Beach, FL (US); Howard Steven Klymas, Panama City Beach, FL (US)

(72) Inventors: Joe Denton Brown, Panama City Beach, FL (US); Howard Steven Klymas, Panama City Beach, FL (US)

(21) Appl. No.: 14/520,551

(22) Filed: Oct. 22, 2014

Related U.S. Application Data
(63) Continuation-in-part of application No. 14/218,407, filed on Mar. 18, 2014.
(60) Provisional application No. 61/894,393, filed on Oct. 22, 2013.
(60) Provisional application No. 61/787,599, filed on Mar. 15, 2013.
(60) Provisional application No. 61/824,755, filed on May 17, 2013.

(51) Int. Cl. A61B 18/24

(52) U.S. Cl. CPC A61B 18/24; A61B 2018/2205
USPC 606/15

(57) ABSTRACT

A method for positioning a surgical laser fiber to facilitate delivery of therapeutic light to a tissue utilizes a flexible insertion sleeve to protect a scope as the fiber and sleeve are guided to a treatment site past bends in the scope. The fiber and sleeve are fixed relative to the scope by a fiber lock. The sleeve may include holes and/or other optional features to protect the fiber or scope during insertion and treatment.

This application claims the benefit of provisional U.S. Patent Appl. Ser. No. 61/894,393, filed Oct. 22, 2013, and incorporated herein by reference.

This application is a continuation-in-part of copending U.S. patent application Ser. No. 14/218,407, filed Mar. 18, 2014, and incorporated herein by reference, which claims the benefit of provisional U.S. Patent Appl. Ser. Nos. 61/787,599, filed Mar. 15, 2013, and 61/824,755, filed May 17, 2013.

### BACKGROUND OF THE INVENTION

1. Field of the Invention

This invention relates to a method for positioning a surgical laser fiber to facilitate delivery of therapeutic light to a tissue, the method including the steps of (a) guiding an introducer or “scope” to a treatment site, the scope being deflected as necessary to reach the treatment site (this step may be carried out before or after steps (b) and (c); (b) inserting a surgical laser fiber into a flexible introducer sleeve to a desired position relative to the distal end of the introducer sleeve (i.e., the end to be situated at the treatment site) based on markings on the fiber; (c) locking the fiber at a desired position within the flexible introducer sleeve using a fiber lock at a proximal end of the introducer sleeve; (d) inserting the flexible introducer sleeve and fiber into the deflected scope and guiding the sleeve to the treatment site; (e) connecting the fiber lock to the scope, thereby positioning the fiber relative to the end of the scope; and (f) optionally mechanically adjusting the position of the fiber and/or sleeve after securing the fiber lock to the scope.

The invention also relates to a surgical laser fiber positioning arrangement that includes a flexible protective introducer sleeve into which the fiber is inserted before insertion into the scope, the flexible introducer sleeve including a fiber lock for locking the fiber at the proximal end of the scope (i.e., the end opposite the treatment end), the flexible introducer sleeve protecting the scope from scoring and punctures during insertion of the fiber into the scope, and the fiber lock enabling accurate and quick positioning of the distal end of the fiber relative to the end of the introducer sleeve and scope, based on markings on the fiber that are visible at the proximal end of the scope.

In addition to facilitating positioning of the fiber, the flexible introducer sleeve may optionally be arranged to include features that insulate the fiber from surrounding cooling fluids, maintain adequate fluid flow through and past the sleeve, and serve as an indicator of overheating to enable early detection of excess heating or burning of tissues or equipment at the treatment site. Further optional features of the flexible introducer sleeve are described in the Detailed Description of Preferred Embodiments section below.

2. Description of Related Art

A disadvantage of current systems that utilize a ureteroscope or other endoscopic apparatus to guide a laser surgical fiber to a treatment site is that the relatively sharp-edge tip of the fiber may cause scoring or punctures in the inner wall of the scope as the fiber is inserted to the treatment site, particularly at deflected sections or bends in the scope. Many ureteroscopic applications, including those involving the kidneys, require bends of greater than 180 degrees (and possibly up to 220 degrees), making it virtually impossible to insert the fiber into the deflected scope without damage, and thereby necessitating insertion of the fiber into the scope before insertion of the scope into the patient. As a result, the ability of the scope to deflect is either limited by the thickness of the fiber, or the size of the fiber must be limited to ensure that the scope can be sufficiently deflected.

One possible approach to the problem of scope damage is to form the tip of the fiber into a rounded or ball shape, but a rounded tip cannot deflect the laser beam in a particular direction and is not optimal for many applications. In addition, the rounded tip may still limit the size of the fiber, impede insertion of the fiber into the scope, or affect the flow of irrigation fluid past the fiber.

An alternative to the rounded fiber tip approach has been to provide surround the fiber with a fixed protective sheath that protects the inner wall of the scope's working channel. One such protective sheath is discussed in the inventor's copending U.S. patent application Ser. No. 13/127,911, filed May 5, 2011 (based on PCT Appl. No. PCT/US2009/006021) and the inventor's copending PCT Appl. No. PCT/US2009/006021, filed Nov. 6, 2009. The present invention replaces the previously proposed protective sheath with a flexible insertion sleeve, and modifies corresponding apparatus and implementation methods, to facilitate insertion and positioning of the fiber in a deflected scope. In addition to protecting the working channel of the scope, the flexible insertion sleeve may include features of the previously proposed protective sheath, such as insulation and overheating protection, as well as additional modifications that provide advantages after insertion of the fiber and during treatment.

### SUMMARY OF THE INVENTION

It is accordingly a first objective of the invention to provide an improved method and apparatus for positioning a surgical laser fiber within a scope or introducer to facilitate delivery of therapeutic light to a tissue.

It is a second objective of the invention to provide a method for positioning a surgical laser fiber, with minimal damage to the scope or introducer through which the fiber is inserted, and without unduly limiting fiber size or the angle to which the scope can be deflected.

It is a third objective of the invention to provide an apparatus that enables positioning of a surgical laser fiber to facilitate delivery of therapeutic light to a tissue, with minimal damage to a scope or introducer through which the fiber is inserted, and without unduly limiting fiber size or the angle to which the scope can be deflected.

It is a fourth objective of the invention to provide an improved introducer sleeve or protective sheath that surrounds a laser delivery fiber during insertion of the fiber into a scope, protects the fiber during surgery, and optionally includes additional features that protect the fiber, other equipment, or patient, and/or that assist the operator/clinician or otherwise facilitate a treatment procedure.

In one aspect of the invention, the objectives are achieved by a surgical laser fiber positioning method that includes the steps of:

(a) guiding an introducer or “scope” to a treatment site, the scope being deflected as necessary to reach the treatment site;

(b) inserting the fiber into a flexible introducer sleeve to a desired position relative to the end of the introducer sleeve based on markings on the fiber;

(c) locking the fiber at a desired position within the flexible introducer sleeve using a fiber lock at one end of the introducer sleeve;

(d) inserting the flexible introducer sleeve and fiber into the deflected scope and guiding the sleeve to the treatment site; and

(e) connecting the fiber lock to the scope, thereby positioning the fiber relative to the end of the scope. In addition, the method of the invention may include an optional step, if the apparatus of the invention is equipment with an adjustment mechanism, of mechanically adjusting the position of the fiber and/or sleeve after locking of the fiber to the sleeve.

It will be appreciated by those skilled in the art that the above-listed steps are not necessarily limited to a particular order, and in particular that steps (b) and/or (c) may be performed before step (a).

The method of the invention is implemented, according to a further aspect of the invention, by providing a fiber-fixing connector that locks the fiber at a desired position in the flexible introducer sheath, that is fixed to the first or proximal end of the scope after insertion of the fiber and sleeve into the scope, and that may further include an adjustment mechanism that enables fine adjustment of fiber and or sleeve position after locking of the fiber. Positioning of the fiber is preferably facilitated by markings on the fiber that are located at predetermined lengths from the distal end of the fiber to enable the operator or physician to easily determine the exact position of the fiber relative to the proximal end of the flexible introducer sleeve or scope, before locking of the fiber to the sheath and connection to the scope. In a preferred embodiment of the invention, the connector is a Luer connector.

Optionally, the flexible introducer sleeve may include holes to allow the physician to maintain adequate flow both through and around the sheath, thereby permitting the sheath to have an internal diameter (ID) that is close to the maximum outer diameter (OD) of the buffer of the optical fiber, In another version, a larger ID may be provided to allow for the introduction of larger diameter devices such as a 2.4 Fr basket. In this case, the holes provide a significant amount of additional flow through the sheath when the smaller diameter laser is used through the larger sheath. This improvement of providing holes in the sheath is also useful for sterilization of the sheath/fiber assembly since the holes allow for a much shorter path for the ETO sterilizing gas to reach all portions of the sheath/fiber assembly, thereby allowing a less robust (and cheaper) sterilization cycle to be used or providing a larger margin of safety to prevent occasional sterilization failures. This improvement may be used with or separately from the fiber-positioning method and apparatus described above.

In addition to positioning markings on the fiber itself, visible markings may optionally also be included on the flexible introducer sleeve for use as trim indicators, and on devices other than the fiber, such as a basket, to enable quick exchange and positioning of the fiber and other devices during a treatment procedure.

Still further, the connector that fixes the fiber to the flexible introducer sleeve in the above-described fiber-positioning apparatus may include a sealing mechanism that allows the operator or physician to lock the fiber's position relative to the scope and also seals the pressurized zone inside the working channel from the unpressurized zone outside of the working channel thereby preventing leakage from the working channel. This sealing mechanism can take several forms. A Touhy-Borst connector that has a male Luer lock connector can be locked to the female Luer lock end of the universal connector provided with the sleeve that is already locked to the scope. This connector can then be tightened onto the laser fiber to lock relative positions and provide a leak proof seal. Alternatively a pin vise may be locked both to the fiber-locking Luer lock connector and then locked onto the laser fiber providing a leak-proof seal. Still further, a third sealing mechanism may consist of a septum-type seal that is pierced by the laser fiber or other instrument in the working channel and snaps easily over the sleeve's female Luer lock connector already attached to the working channel. When the septum is pierced by the laser fiber, the flexible portion of the septum seals against the fiber preventing leakage past the seal, yet also locks the fiber's position relative to the sleeve.

These and other improvements will be described in greater detail below in connection with the accompanying drawings, which show preferred embodiments of the invention.

It will be appreciated that the term “scope” as used in the following description may refer to a ureteroscope or other types of endoscopes or introducers through which the fiber may be inserted during a surgical procedure, and that the method and apparatus of the invention is not limited to ureteroscopic applications, but rather may be used in connection with other laser surgery applications.

### BRIEF DESCRIPTION OF THE DRAWINGS

FIG. 1 is a flowchart showing a fiber positioning method according to a preferred embodiment of the invention.

FIGS. 2 and 3 are side views showing an apparatus for implementing the method of FIG. 1.

FIG. 4 is a side view of a variation of the apparatus of FIGS. 2 and 3.

FIG. 5 is an end view of the apparatus of FIG. 4.

FIG. 6 is a plan view of an introducer sleeve with holes according to an optional variation of the sleeve of FIGS. 2-5.

FIG. 7 is a plan view of the sheath of FIG. 6 with a locking device to hold the position of the fiber relative to the sheath.

FIG. 8 is a plan view of a sheath and fiber with a position indicator.

FIG. 9 is a plan view of the sheath and fiber of FIG. 8 with a pin vise to lock the fiber position.

FIG. 10 is a plan view of a sheath arrangement with a dilator and a coating for pull-in or fiber breakage detection.

FIG. 11 is a plan view of one embodiment of the fiber tip and sheath position relative to the end of the endoscope when the fiber is ready to treat tissue.

FIG. 12 is an exploded plan view of an alternative embodiment of the universal Luer lock connector, with a seal.

FIG. 13 is an actual photo of one embodiment of the Luer lock connector, seal and marking on fiber relative to the Luer lock connector.

FIG. 14 is a plan view of one preferred embodiment of what the two sets of marks will look like on a laser fiber.

### DETAILED DESCRIPTION OF THE PREFERRED EMBODIMENTS

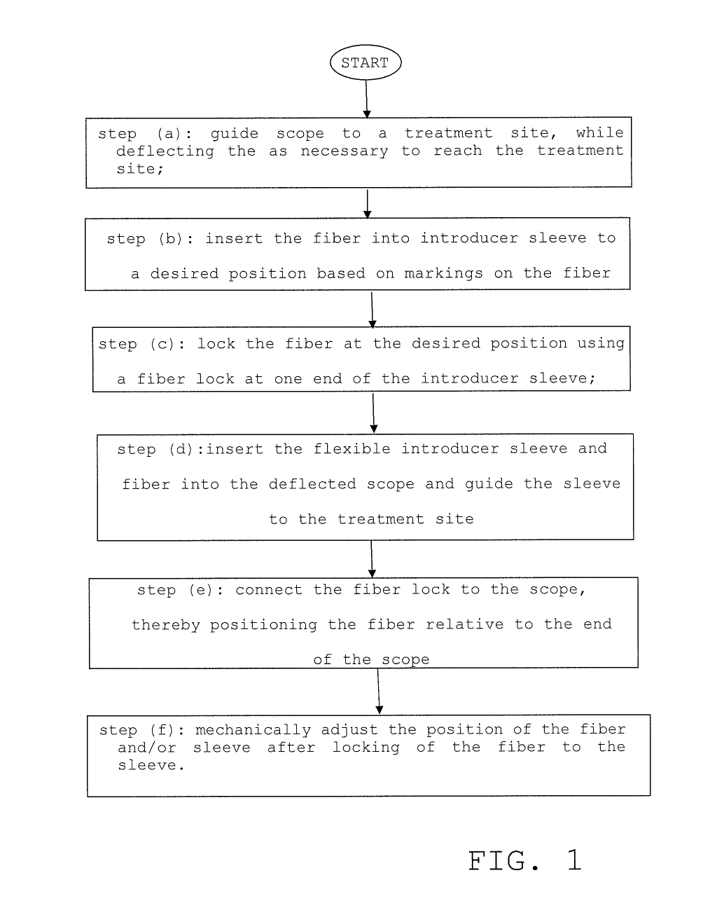

FIG. 1 is a flowchart of the basic positioning method of a preferred embodiment of the invention.

As illustrated in FIG. 1, the method includes the step (a) of guiding an introducer or “scope” to a treatment site, the scope being deflected as necessary to reach the treatment site. In step (b), which may occur before or after step (a), the surgical laser fiber is inserted into a flexible introducer sleeve to a desired position relative to the distal end of the introducer sleeve (i.e., the end to be situated at the treatment site) based on markings on the fiber that are positioned at predetermined distances from the end of the fiber, and that indicate the distances, for example by the number or spacing of the markings. In step (c), the fiber is locked at a desired position within the flexible introducer sleeve using a fiber lock situated at the proximal end of the introducer sleeve. The fiber lock may utilize a Luer connector to hold the fiber and introducer sleeve, and thereby lock the fiber in position relative to the sleeve during insertion of the fiber and sleeve into the scope. In step (d), the flexible introducer sleeve and fiber are inserted into the deflected scope and guided past any bends in the scope to the treatment site. At this point, the fiber lock may be connected or coupled in step (e) to the scope, thereby positioning the fiber relative to the end of the scope. If the fiber lock is provided with an optional adjustment mechanism, the position of the fiber and/or sleeve relative to the scope may be further adjusted even after securing the fiber lock to the scope.

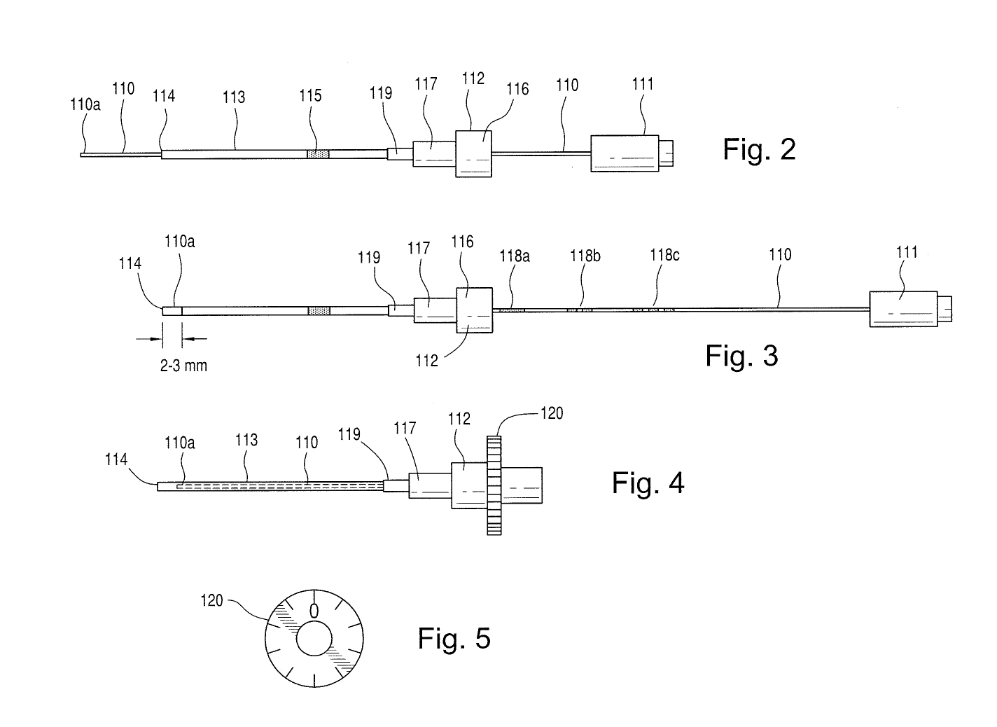

The system or apparatus by which the method of FIG. 1 is carried out is illustrated in FIGS. 2-5. The fiber itself is indicated by reference numeral (110) and is illustrated as being coupled at a first end to a laser via a standard coupler (111). As shown in FIG. 2, the fiber (110) has been inserted through a fiber lock (112) and into a flexible introducer sleeve (113) such that the fiber is positioned near the distal end (114) of the sleeve. As shown in FIG. 3, final positioning of the fiber tip (10a) relative to the end (114) of the sleeve is achieved with reference to different sets of markings (118a, 118b, 118c) at the proximal end of the fiber. As shown in FIG. 3, by way of example and not limitation, the desired position of the fiber tip (110a) may be within the sleeve at a distance of 2-3 mm from the distal end (114) of the sleeve, indicated at the proximal end of the fiber (110) by fiber marking (118a). If the desired position of the fiber tip (110a) is beyond the end of the scope, markings (118b) or (118c) could be used. The sleeve (113) may include optional deflection indicators or sensors (115) for use during insertion into a scope. The fiber lock (112) includes a mechanism (116) for securing or locking the fiber with respect to the introducer sleeve (113), and also a coupling section (117) for securing the fiber lock (112) to the scope. FIGS. 2 and 3 only show a small section (119) of the scope, but it will be understood that when the fiber (110) and flexible introducer sleeve (113) are inserted into the scope, the scope will extend to the left of the fiber connector (112) as shown in FIGS. 2 and 3.

FIGS. 4 and 5 show a variation of the embodiment of FIGS. 2 and 3, in which an optional adjustment mechanism (20) is included on the fiber lock (112) to enable fine adjustment of the position of the fiber (110) relative to the sleeve (113), and/or the position of the fiber (110) and/or sleeve (113) relative to the scope, after locking of the fiber (110) to the sleeve (113) or after coupling of the fiber lock (112) to the scope. As illustrated, the adjustment mechanism (120) is in the form of a rotatable adjustment wheel, but any other mechanically-equivalent adjustment mechanism may be substituted.

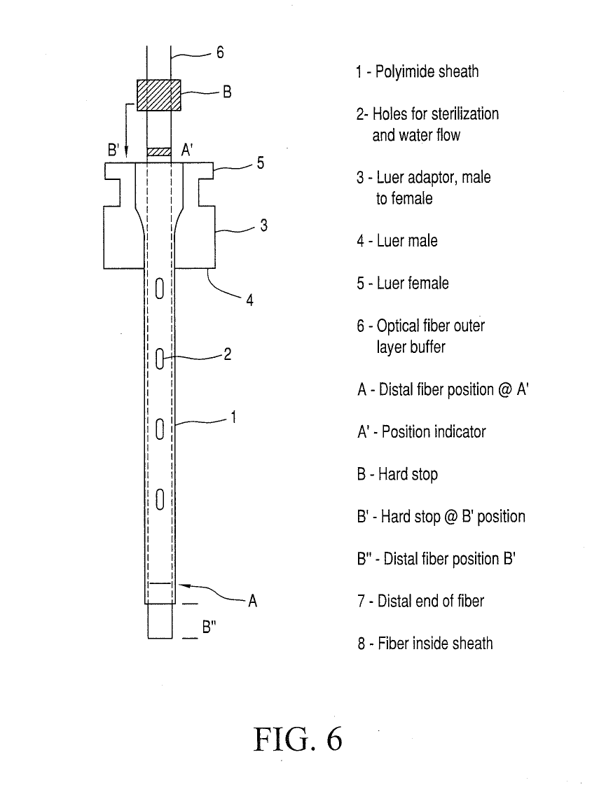

FIG. 6 shows an alternative sleeve or sheath (1) that include holes (2), and that may optionally be used in addition to or in place of the flexible introducer sleeve of the preferred embodiments illustrated in FIGS. 2-5, or in connection with systems or apparatus other than those of the preferred embodiments of FIGS. 2-5. As shown in FIG. 6, the sleeve or sheath including holes (2) is a very thin-walled polyimide sheath (1) having an inner diameter (ID) that is very close to the maximum outer diameter (OD) of the optical fiber buffer of an optical fiber (6), thus providing maximum room for flow when inserted into a scope. Another design variation utilizes a larger ID that allows for the introduction of larger diameter devices such as a 2.4 Fr basket. In this case the holes (2) in the sheath (1) allow the physician to maintain adequate water or fluid flow both through and around the sheath that would not be available with a larger OD sheath without holes. Another potential use for this sheath is to provide for the injection of an addition fluid or gas at the very tip of the fiber.

A liquid dye that absorbs a laser wavelength extremely well could be injected through the fiber sheath to enhance the stone breaking properties of a given laser wavelength. Dye could be injected just prior to laser pulse through the coaxial sheath to ensure maximum concentration exactly at the fiber tip and also to minimize dye concentrations in other areas away from the fiber tip where the dye may reduce visibility of the physician. Dye may be able to be attached to another compound that has an affinity for and sticks to stones which will also help ensure a high concentration of dye very near the stone and minimize loss of visibility. A high viscosity dye may also be utilized to help maintain a high concentration near the stone and reduce tendency to become rapidly diluted in the irrigating flow. This technique may allow cheaper diode laser wavelengths such as 1470 nm to be used to break stones. The sheath connector that has the capability to adjust the position of the sheath relative to the scope will allow the dye to be injected immediately adjacent to the stone.

In addition to providing enhanced fluid flow, the holes (2) facilitate sterilization. Having an extremely small clearance between the ID of the sheath and the OD of the fiber over a very long length presents a major challenge to sterilizing this product. The addition of one or more small holes (2) to the sheath along the length of the sheath provides a greatly enhanced access of the sterilizing gas/agent to the entire length of the sheath/fiber assembly. In addition, these holes may be utilized in some designs to also provide fluid flow through the space between the fiber and sheath to augment the flow around the OD of the sheath.

FIG. 6 also shows a luer lock connector (3) attached to the reinforced section of the sheath. This universal luer lock (3) has a male luer connector (4) at its distal end to allow the physician to lock the sheath to the female luer lock at the entrance to the scope's working channel. When the luer lock is attached to the scope, the sheath will be precisely positioned extending approximately 2 mm (or any desired distance) from the end of the ureteroscope's working channel. The female luer lock connector (5) at the proximal end allows for subsequent attachment of a pin vise Touhy-Borst or a septum-type seal if the physician desires to lock the position of the fiber to the sheath during insertion, use or removal of the fiber/sheath assembly. This seal also functions to prevent leakage out of the working channel.

In order to assist the physician in positioning the distal end of the fiber, at least one position indicator A′ (corresponding to markings (18a, 18b, and 18c of the first preferred embodiment) may be provided on the fiber. The correct position (A) of the fiber tip relative to the distal end of the sheath during insertion or removal of the sheath/fiber assembly from the scope's working channel is established when the position indicator (A′) is correctly positioned flush to the proximal entrance to the sheath. This ideal fiber tip position (A) may be, in the case of a urological application, approximately 1-2 mm recessed inside the sheath.

As is also the case with markings (18a, 18b, and 18c) of the embodiment of FIGS. 2-5, the width of this mark may be a precise width that will allow the front edge of the mark to be lined up at the entrance to the working channel of the scope to indicate the recessed fiber tip position, while the rear edge of the mark may be used to provide an indicator of the approximate position of the fiber tip when it is in an extended position approximately 1.5 mm in front of the sheath ready to treat tissue. This allows the operator or clinician to confidently extend the fiber out of the sheath without looking into the scope. Then when he does look into the scope, he can fine-tune the position of the already visible fiber tip to his individual preferred treatment extension length. To make provision for the event of the fiber needing to be re-cleaved during a procedure, a series of matched marks can be printed on the laser fiber at the distal tip and at the entrance to the scope as detailed above. These two sets of marks will be precisely positioned so that the fiber can be stripped and re-cleaved at the distal mark allowing the corresponding second mark to be used to position the fiber precisely at the entrance to the working channel of the scope ensuring that the re-cleaved fiber tip will again be positioned 1 mm recessed into the sheath during insertion and removal from the scope. The buffer stripping equipment provided with the fiber for re-cleaving will be designed to function with the distal marks to ensure correct placement when utilized with the corresponding proximal mark. Double, triple, etc., sets of marks may be provided to allow for a corresponding number of re-cleaving operations. FIG. 9 demonstrates one possible version of matching sets of marks.

Distance (A) is based on physician feedback as to the average or maximum length of fiber that is removed during each re-cleaving operation, e.g. 10 mm.

In addition to marking the advance of the distal end (7) of the fiber (8) from the sheath (1), markings may be used to indicate that the distal end (7) of the fiber is safely within the sheath. This solves the problem that there is a blind spot between the working position (A) of the fiber and the end of the scope (9), within which the exact position of the fiber is difficult to determine because the tip cannot be seen. The blind spot is illustrated in FIG. 1A and indicated by reference numeral (10), which refers to a distance between the end of the scope and a distance at which the advancing fiber enters the visibility cone (12) visible from the scope. When the fiber tip (7) is within the region indicated by reference numeral (10), it cannot be seen. When the fiber tip is advanced into the region indicated by reference numeral (11), then it is within the visibility cone (12).

Distance (B′) is based on the length of the sheath designed for the working channel of a particular scope brand and/or model, e.g. 850 mm for the Storz ×2 flexible ureteroscope. This length of the sheath will accommodate the variability in scope length of about ±2 mm by centering its target length at the point where the sheath tip is flush with the scope's tip or about lmm recessed into the scope. This will ensure that the sheath does not extend too far beyond the tip of the scope. Although the ideal placement of the sheath has the sheath just visible in the field of view, which is about 1 to 1.5 mm beyond the scope's tip, the sheath can be positioned just out of the field of view with no reduction in protection of the working channel's lining.

Distance (B″) is based on the difference between the recessed fiber position (about 1 to 1.5 mm recessed into the sheath) and the protruding fiber position after it has been advanced to treat stones or tissue (about 1 to 2 mm in front of the end of the sheath). Therefore, this distance (B″), approximately 2 to 3.5 mm, will allow the physician to use the front edge to indicate recessed position for sheath insertion/removal while the rear edge of mark will indicate the protruding position where the fiber is ready for use. The number of marks may be set to allow three re-cleaving procedures for a single use disposable, while a significantly larger number of marks may be provided for a multiple use fiber. The number of marks may be used to help ensure that a fiber is not used more than prudent number of times. Marks can be matched using a plurality of lines, different colors or shapes to enable the physician to determine which external mark to use after each re-cleaving operation.

Finally, as shown in FIG. 6, a hard stop (B) can be provided to prevent the laser fiber from extending too far past the end of the sheath as a safety mechanism to prevent damage to non-target tissue. The fiber can be advanced into the sheath until the hard stop (B) reaches the position (B′) where it is physically prevented from advancing any further due to its larger diameter not fitting into the sheath. The advance position of the distal tip of the fiber out of the sheath is indicated, as noted above, by B″.

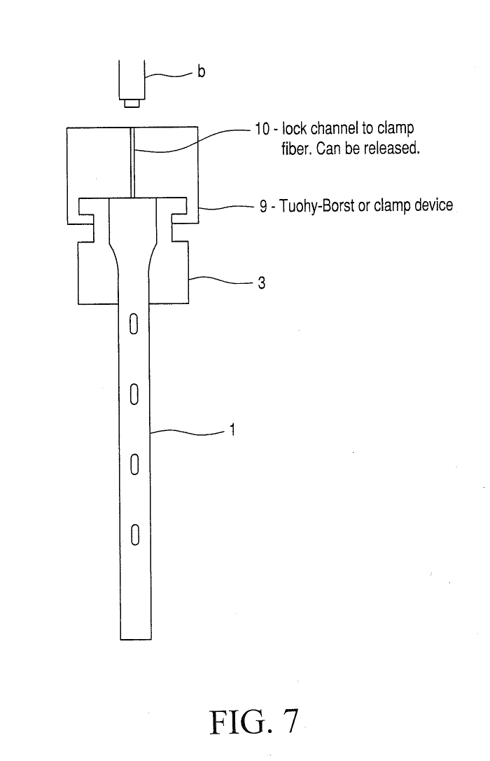

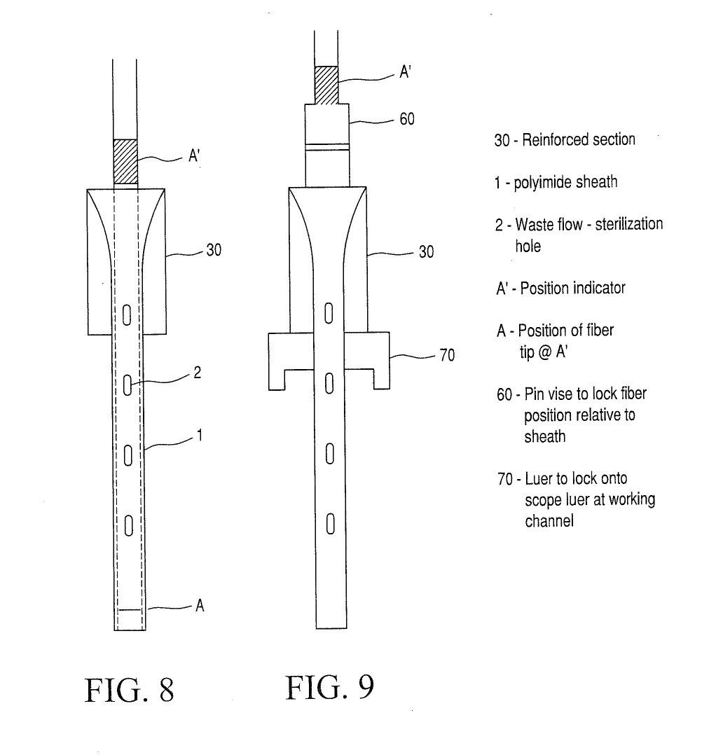

The arrangement shown in FIG. 7 differs from that of FIG. 6 in that a Touhy-Borst device (9), a septum-type seal (11 in FIGS. 7 and 8), or other clamping device such as a pin vise is used to lock the fiber to the sheath during insertion, use or removal of the fiber/sheath assembly. The channel (10) where the fiber is clamped is also shown in FIG. 7.

FIG. 8 shows a variation of the arrangement of FIG. 6, which also includes a very thin-walled polyimide sheath (1) with holes (2) and position indicator A′ corresponding to those of FIG. 1. However, the sheath of this embodiment is further provided with a reinforced section (30) at the proximal end of the sheath, which provides the following advantages. The primary advantage is that it provides a handling means for the physician to handle the extremely fragile polyimide sheath. This prevents kinking and/or breakage during insertion and use of the sheath/fiber assembly. The reinforced section (30) also has a funnel-shaped tip that enhances visibility and facilitates the insertion of the fiber tip into the sheath. Since the reinforced section (30) is short and does not extend past the irrigation side port, it does not reduce the flow around the sheath. A slanted cut on the proximal end of the polyimide sheath (1) minimizes the potential for the advancing fiber to catch on the small ledge formed at the proximal end of the sheath inside the reinforced portion.

FIG. 9 shows a modification of the arrangement of FIG. 8 that includes a pin vise (60) to lock the fiber position relative to sheath. Some physicians may prefer to utilize a pin vise to lock the fiber to the sheath to ensure that they do not move relative to each other during insertion and removal from the working channel of the ureteroscope or other scope. In the case of a urological application, the pin vise (60) may be easily repositioned and re-locked after the fiber has been advanced from its position inside the sheath into the field of view adjacent to the target tissue. The fiber and sheath can be locked together at the desired recess position to allow the physician to take the assembly directly from the package and introduce it safely into the ureteroscope's working channel without having to manually insert the fiber into the sheath or check the position of the fiber relative to the sheath thereby simplifying the procedure.

The arrangement of FIG. 9 also includes a luer lock (70) attached to the reinforced section (30) of the sheath This universal luer lock (70) allows the physician to lock the sheath to a female luer lock at the entrance to the ureteroscope's working channel. When this luer lock is attached to the scope, the sheath will be precisely positioned extending approximately 2 mm from the end of the ureteroscope's working channel. However, a modified mechanism can be provided that allows the physician to adjust the position of the sheath relative to the scope.

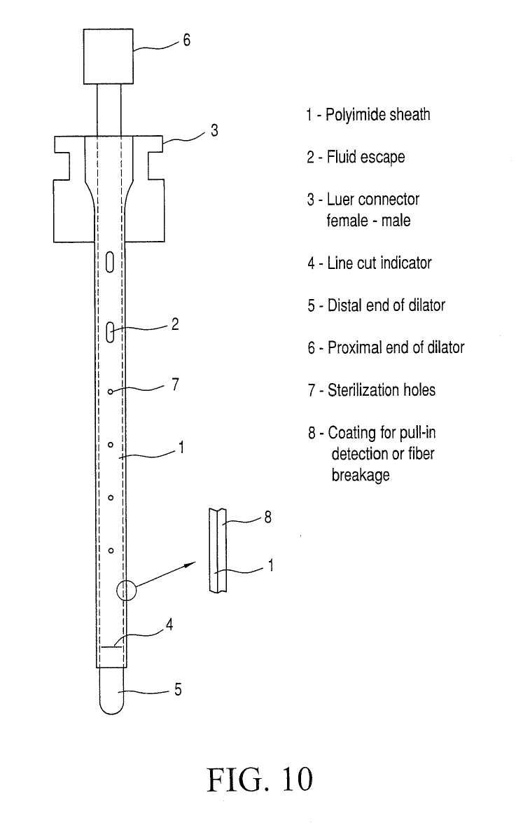

FIG. 10 shows a variation of the sheath (1) of FIG. 6, with an additional line cut indicator or indicators (4). The line cut indicator(s) (4) indicate where the physician should trim the sheath prior to insertion based on specific ureteroscope/connector combinations.

The arrangement of FIG. 10 further includes a dilator with proximal end (5) and distal end (6). The blunt dilator may be provided to allow insertion of the sheath prior to the insertion of the fiber. The distal blunt end (5) of the dilator would extend approximately 1 mm past the end of the sheath to prevent damage to the inner lining of the scope's working channel. The distal end (6)of the dilator may be provided with a male luer lock connection that would allow it to lock to the sheath during insertion. The dilator provides stiffening of the sheath to prevent kinking and damage to the sheath during insertion and removal from working channel.

The sheath (1) shown in FIG. 10 further includes a coating (8) for detection of fiber breakage or pull-in to sheath. The coating (8) is layered into or on the sheath to provide a distinct signal that enables the safety mechanism detector to differentiate between standard stone treatment pulses and pulses that occur if the fiber breaks or if the fiber is accidentally pulled into the sheath. If either of these occurs, the safety mechanism will recognize the distinct signal and shut the laser down prior to any damage to the ureteroscope's working channel. There are many ways that this coating may work. One instance is incorporation of Holmium coating that is activated by the Holmium laser that is treating the stones. When it is activated by that wavelength it will re-emit a much longer signal that can be readily differentiated from the Holmium treatment pulse and activate the safety shutdown.

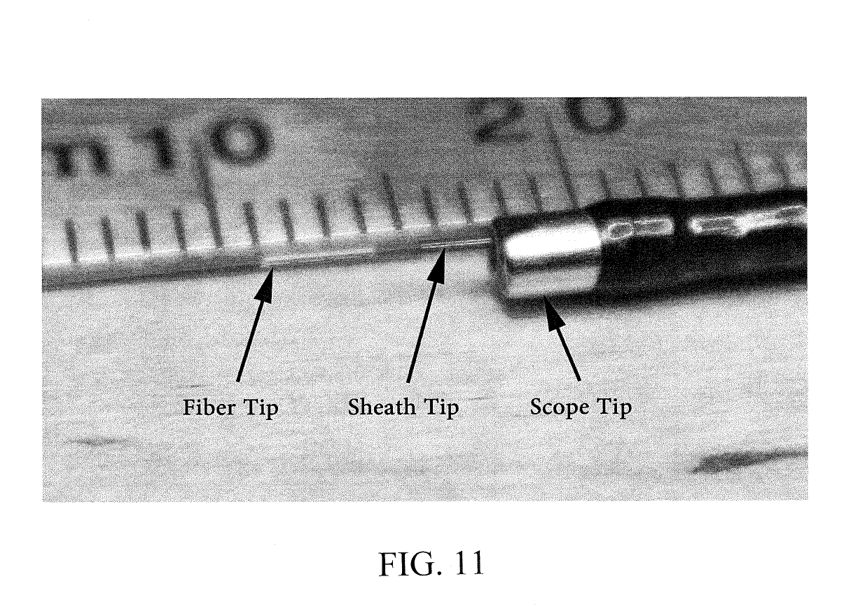

FIG. 11 demonstrates the sheath and fiber position relative to the distal end of the working channel of the ureteroscope. The sheath extends approximately 2 mm from the end of the scope. The fiber tip extends an additional distance beyond the end of the sheath to treat the stones and/or tissue without damaging the sheath or scope.

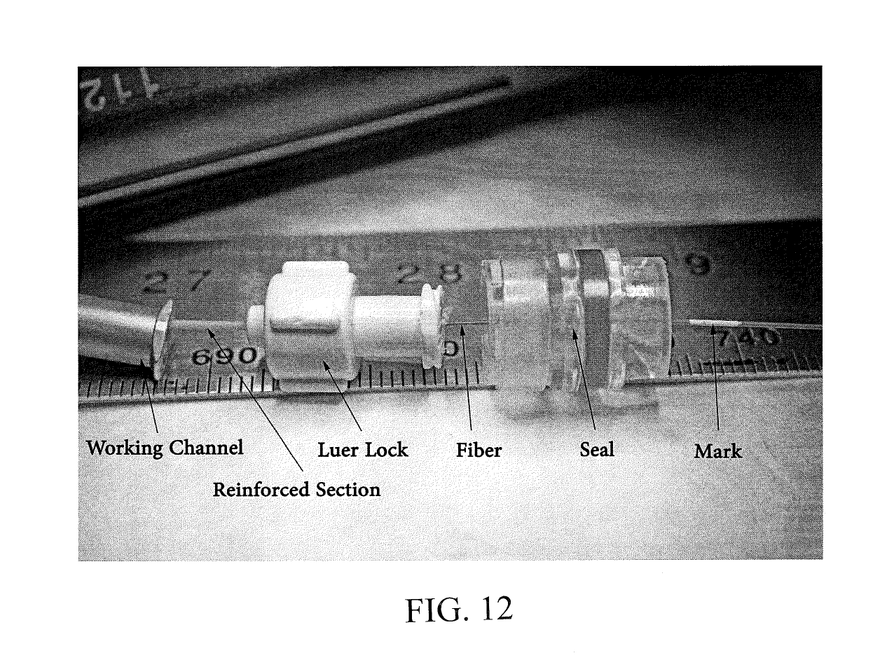

FIG. 12 demonstrates the exploded view of the components, Universal Luer lock connector and seal (11), that attach to the scope and fiber at the proximal end of the working channel of the scope.

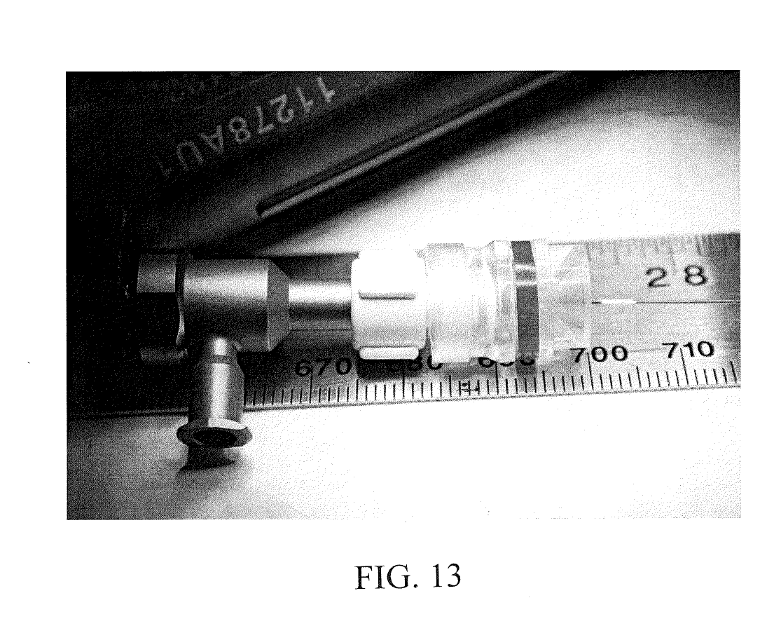

FIG. 13 demonstrates an actual view of the seal and Luer lock connector attached to the stainless steel female Luer lock connector of the ureteroscope.

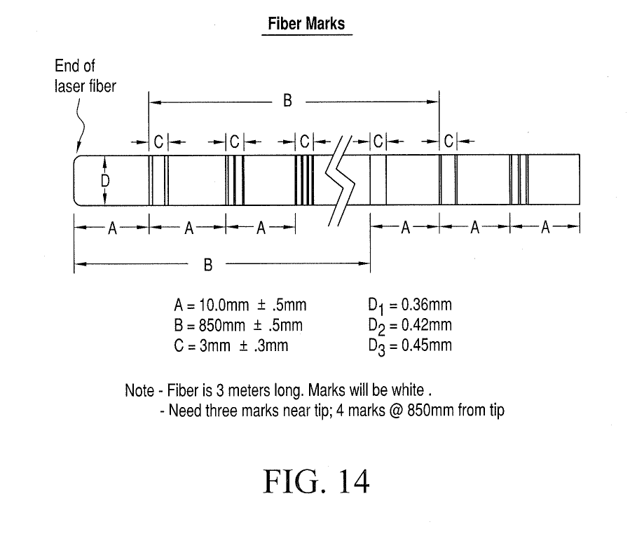

FIG. 14 shows details of a possible version of the two sets of laser fiber markings that allows re-cleaving of the laser fiber during treatment.

I claim:

1. A method of positioning a surgical laser fiber in a scope, comprising the steps of:
(a) guiding scope to a treatment site, the scope being deflected as necessary to reach the treatment site;
(b) inserting the fiber into a flexible introducer sleeve to a desired position relative to the end of the introducer sleeve based on markings on the fiber;
(c) locking the fiber at the desired position within the flexible introducer sleeve using a fiber lock at one end of the introducer sleeve;
(d) inserting the flexible introducer sleeve and fiber into the deflected scope and guiding the sleeve to the treatment site; and
(e) connecting the fiber lock to the scope, thereby positioning the fiber relative to the end of the scope. of mechanically adjusting the position of the fiber and/or sleeve after locking of the fiber to the sleeve.

2. A method as claimed in claim 1, further comprising the step of mechanically adjusting the position of the fiber and/or sleeve after locking of the fiber to the sleeve

3. Apparatus for positioning a surgical laser fiber in a scope, comprising:
a flexible introducer sleeve;
a fiber-locking connector that locks the fiber at a desired position in the flexible introducer sheath, and that is also arranged to be fixed to a proximal end of the scope after insertion of the fiber and sleeve into the scope.

4. Apparatus as claimed in claim 3, wherein the fiber-locking connector further includes an adjustment mechanism that enables fine adjustment of fiber and/or sleeve position after locking of the fiber.

5. Apparatus as claimed in claim 3, wherein the fiber-locking connector is a Luer connector.

6. Apparatus as claimed in claim 3, further including markings on the fiber situated a predetermined distance from a treatment end of the fiber for positioning the fiber relative to a proximal end of the flexible introducer sleeve.

7. A protective sheath for an optical fiber used in surgical laser procedures, wherein the protective sheath includes a plurality of holes for improved fluid flow and sterilization.

8. A protective sheath as claimed in claim 7, wherein inner diameter of the sheath is close to a maximum outer diameter of the a buffer of the optical fiber.

9. A protective sheath as claimed in claim 7, wherein an inner diameter of the sheath is larger than an outer diameter of a fiber buffer to allow for introduction of larger diameter devices.

10. A protective sheath for an optical fiber used in surgical laser procedures, further comprising a luer connector for locking the sheath to the working channel of an introducer or scope.

11. A protective sheath for an optical fiber used in surgical laser procedures, further comprising one or more sets of markings at either just the proximal end of the fiber or at both the proximal end of the fiber to facilitate positioning of a distal end of the fiber relative to a distal end of the sheath. The markings at the distal end of the fiber allows the physician to cleave the fiber at the precisely correct position that allows the corresponding mark at the proximal portion of the fiber to be used in positioning the fiber tip recessed into the sheath during insertion and removal from the endoscope.

12. A protective sheath as claimed in claim 11, further comprising trim markings at a distal and/or proximal end of the fiber for facilitating cutting of the sheath to a desired length.

13. A protective sheath as claimed in claim 11, wherein the number of sets of markings on the matched laser fiber is at least two, to facilitate positioning after re-stripping.

14. A protective sheath for an optical fiber used in surgical laser procedures, wherein a proximal end of the sheath is reinforced to facilitate securing of the optical fiber to the sheath by a securing device.

15. A protective sheath as claimed in claim 14, wherein the securing device is a pin vise.

16. A protective sheath as claimed in claim 14, wherein a universal luer lock connector is attached to the reinforced section of the sheath for securing the sheath to an introducer or scope.

17. A protective sheath for an optical fiber used in surgical laser procedures, further comprising a hard stop provided to prevent a laser fiber from extending too far past an end of the sheath.
